(19) United States
(12) Patent Application Publication
(10) Pub. No.: US 2019/0275344 A1
(43) Pub. Date: Sep. 12, 2019

(54) PLASMA DISCHARGED SKIN TREATMENT DEVICE

(71) Applicant: THE WORKS, INC., Incheon (KR)

(72) Inventor: Hyoung Suk KIM, Siheung-Si, Gyeonggi-Do (KR)

(21) Appl. No.: 16/063,996

(22) PCT Filed: Apr. 20, 2017
(86) PCT No.: PCT/KR2017/004214

§ 371 Date: Jun. 19, 2018

(30) Foreign Application Priority Data
Dec. 22, 2016 (KR) 10-2016-0176260

(51) Int. Cl. A61N 1/44; H05H 1/24; A61M 37/00

(52) U.S. Cl. CPC A61N 1/44; H05H 1/2406; A61M 2037/0007; H05H 2001/2412; A61M 37/00

(57) ABSTRACT

The present invention provides a plasma discharged skin treatment device. The plasma discharged skin treatment device of present invention includes a first insulator, a first electrode provided on one surface of the first insulator, a second insulator provided on one surface of the first electrode, a second electrode provided on one surface of the second insulator, and a third insulator provided on one surface of the second electrode to face a skin of a user, wherein a plasma is generated by a power applied between the first electrode and the second electrode. According to the present invention, there is provided a large-area plasma discharged skin treatment device which is capable of being flexibly deformed along a facial surface of a user while allowing to be semipermanently used. In addition, according to the present invention, there is provided a large-area plasma discharged skin treatment device which is capable of enhancing effects of moisturizing a skin and penetrating a drug into the skin through skin stimulation caused by visible rays, infrared rays, plasma ions, and active species generated from plasma, and allowing a wound to be treated on a skin portion of a body such as a neck, waist, arm, leg and the like when using the device on the corresponding portion.

### TECHNICAL FIELD

The present invention relates to a plasma discharged skin treatment device, and more specifically, to a plasma discharged skin treatment device capable of being flexibly deformed along a facial surface of a user while allowing to be semipermanently used, as well as, enhancing effects of moisturizing a skin and penetrating a drug into the skin through skin stimulation caused by visible rays, infrared rays, plasma ions, and active species generated from plasma, and allowing a wound to be treated on a skin portion of a body such as a neck, waist, arm, leg and the like when using the device on the corresponding portion.

### BACKGROUND ART

Most of mask packs, which are widely used for cosmetic purposes, are a disposable product, and are discarded after one use. Meanwhile, recently released LED masks are products which can be used repeatedly and are adapted to compensate disadvantages of the disposable mask pack. However, there is a technical limitation that a form of the LED mask cannot be deformed so as to fit a face shape of an individual user.

In addition, there is also a technical limitation that the LED mask of the related art uses LEDs to emit infrared rays or visible rays, and thereby effects of treating the skin are limited.

### DISCLOSURE

### Technical Problem

Accordingly, it is an object of the present invention to provide a plasma discharged skin treatment device capable of being flexibly deformed along a facial surface of a user while allowing to be semipermanently used, as well as, enhancing effects of moisturizing a skin and penetrating a drug into the skin through skin stimulation caused by visible rays, infrared rays, plasma ions, and active species generated from plasma, and allowing a wound to be treated on a skin portion of a body such as a neck, waist, arm, leg and the like when using the device on the corresponding portion.

### Technical Solution

In order to achieve the above object, according to the present invention, there is provided a plasma discharged skin treatment device, including: a first insulator; a first electrode provided on one surface of the first insulator; a second insulator provided on one surface of the first electrode; a second electrode provided on one surface of the second insulator; and a third insulator provided on one surface of the second electrode to face a skin of a user, wherein a plasma is generated by a power applied between the first electrode and the second electrode.

Preferably, the first electrode has a protrusion formed thereon.

Further, the second electrode is a porous electrode. Furthermore, the first insulator includes polygonal insulation panels which are continuously arranged.

### Advantageous Effects

According to the present invention, there is provided a large-area plasma discharged skin treatment device which is capable of being flexibly deformed along a facial surface of a user while allowing to be semipermanently used.

In addition, according to the present invention, there is provided a large-area plasma discharged skin treatment device which is capable of enhancing effects of moisturizing a skin and penetrating a drug into the skin through skin stimulation caused by visible rays, infrared rays, plasma ions, and active species generated from plasma, and allowing a wound to be treated on a skin portion of a body such as a neck, waist, arm, leg and the like when using the device on the corresponding portion.

### DESCRIPTION OF DRAWINGS

FIG. 1 is a view illustrating a structure of a plasma discharged skin treatment device according to a first embodiment of the present invention.

FIG. 2A is a view illustrating an outer cover structure of a plasma discharged skin treatment device according to a second embodiment of the present invention.

FIG. 2B is a cross-sectional view taken on line B-B′ in FIG. 2A.

FIG. 3 is a cross-sectional view illustrating a structure of a plasma discharged skin treatment device 100 according to a third embodiment of the present invention.

FIG. 4 is a cross-sectional view illustrating a structure of a plasma discharged skin treatment device according to a fourth embodiment of the present invention.

FIG. 5 is a view illustrating a structure of a plasma discharged skin treatment device according to a fifth embodiment of the present invention.

### BEST MODE

Hereinafter, the present invention will be described with reference to the accompanying drawings in detail. Referring to the drawings, wherein like reference characters designate like or corresponding parts throughout the several views. In the embodiments of the present invention, the publicly known functions and configurations that are judged to be able to make the purport of the present invention unnecessarily obscure will not be described.

FIG. 1 is a view illustrating a structure of a plasma discharged skin treatment device 100 according to a first embodiment of the present invention. As illustrated in FIG. 1, referring to a structure of a cut cross-section taken on line A-A′ of any region a in the plasma discharged skin treatment device 100, the plasma discharged skin treatment device 100 includes a first insulator 101 forming an outer layer thereof, a first electrode 102 formed on one surface of the first insulator 101 to serve as an electrode layer, a plurality of second insulators 103 formed on one surface of the first electrode 102 at a predetermined interval, second electrodes 104 provided on one surface of each second insulator 103, and third insulators 105 provided on one surface of each second electrode 104, wherein a plasma is generated by a power applied between the first electrode 102 and the second electrode 104.

In particular, the first insulator 101 is an insulator for securing safety of a user and suppressing unnecessary external discharge, the second insulators 103 is an insulator for preventing the first electrode 102 and the second electrodes 104 from being shorted, and the third insulator 105 is an insulator for preventing a current from flowing to the skin or shielding electromagnetic waves because the first electrode is located adjacent to the skin.

In addition, for embodying the present invention, the first insulator 101, the second insulators 103, the third insulators 105, the first electrodes 102, and the second electrodes 104 may be formed in a thin film form, respectively.

Meanwhile, the third insulator 105 plays a role of an inner surface of the plasma discharged skin treatment device 100, and is a portion to be in contact with a face or a skin of a user. As described above, in the present invention, a principle of a plasma generating device is applied to a cosmetic mask.

Specifically, the plasma generating device refers to a device that generates plasma by a discharge phenomenon of gases including air between two electrodes to which a power is applied. Such a plasma generating device may generate visible rays, infrared rays, ultraviolet rays, as well as plasma ions and plasma active species.

Meanwhile, when applying a power having a frequency of 1 kHz to 100 MHz to the plasma generating device, plasma may be stably generated. When the plasma contacts the skin, there are effects of sterilizing the skin, and improving conditions of the skin by increasing penetration abilities of a moisturizing agent and a drug into the skin. In addition, there are effects of moisturizing the skin, and shortening a recovery period of surgical scars, wound scars, and acne treatment.

Meanwhile, as illustrated in FIG. 1, an adhesive layer 110 that plays a role of an adhesive may be provided between the first insulator 101 forming the outer surface of the plasma discharged skin treatment device 100 and the first electrodes 102.

In addition, the first insulator 101 may be made of a solid-state material including polyimide, polyethylene, and the like, but preferably, made of a flexible insulator including Teflon™, silicone, and the like, such that the first insulator 101 may be flexibly deformed according to a face shape of the individual user to be seated on the face of the user, and thereby enhancing feelings of wearing.

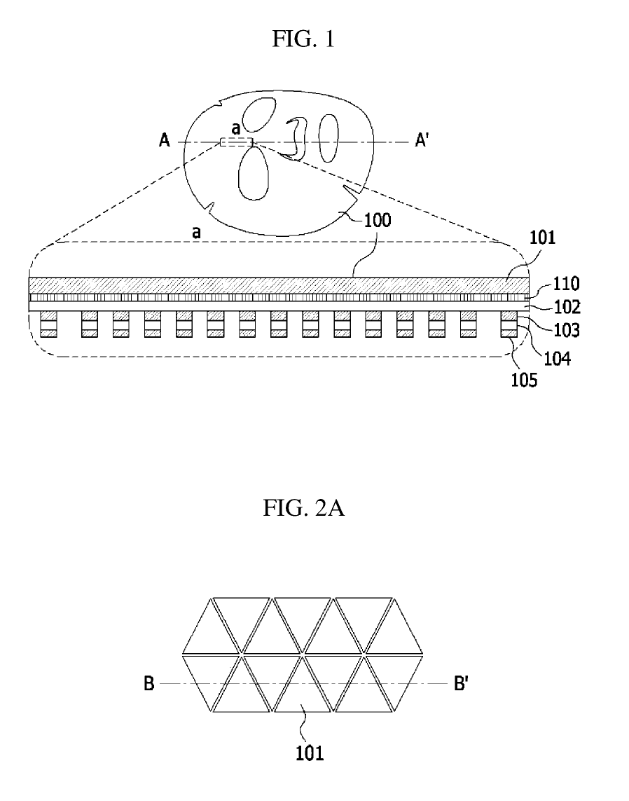

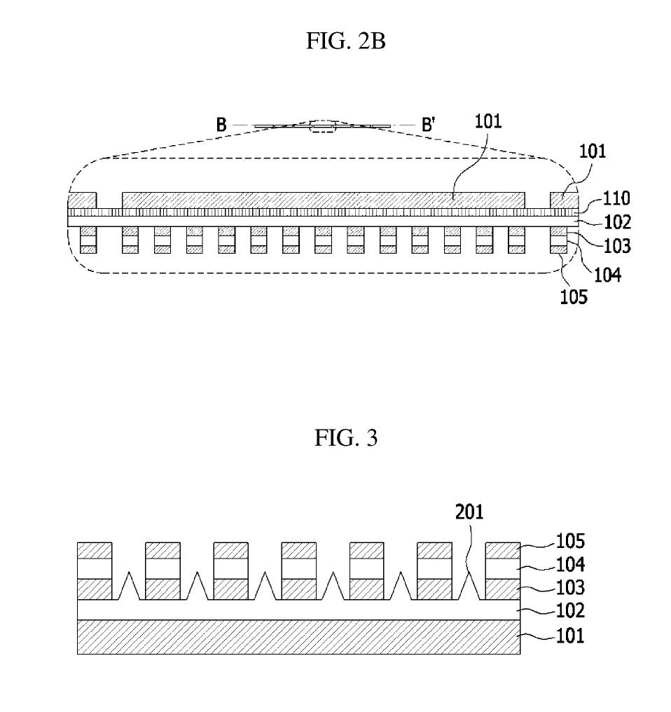

FIG. 2A is a view illustrating an outer cover structure of the plasma discharged skin treatment device 100 according to a second embodiment of the present invention. As illustrated in FIG. 2A, when embodying the present invention, in order to ensure flexibility and variability of the plasma discharged skin treatment device 100, polygonal insulation panels such as a triangle panel may be continuously arranged and connected to each other, thus to implement the first insulator 101 forming the outer cover.

That is, in such a case, even if each of the polygonal insulation panels has a predetermined rigidity, the flexibility and variability of the plasma discharged skin treatment device 100 may be ensured due to the fluidity at portions in which the polygonal insulation panels are connected to each other, and at the same time, external rigidity of the plasma discharged skin treatment device 100 may also be secured.

FIG. 3 is a cross-sectional view illustrating a structure of a plasma discharged skin treatment device 100 according to a third embodiment of the present invention. As illustrated in FIG. 3, second insulators 103 are formed at a predetermined interval on a first electrode 102 formed in layers, and protrusions 201 may be formed on the first electrodes 102 in spaces between the second insulators. Thereby, plasma may be more easily generated by the gas discharge between the first electrode 102 and second electrodes 104 than the case of without protrusion.

Meanwhile, for embodying the present invention, it is preferable that the protrusion 201 has a cone shape so as to facilitate emission of electrons even under a low voltage condition.

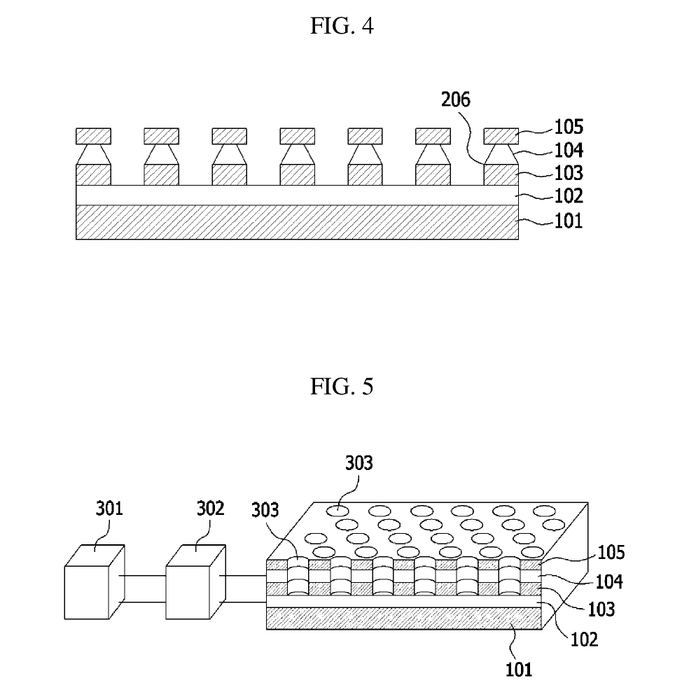

FIG. 4 is a cross-sectional view illustrating a structure of a plasma discharged skin treatment device 100 according to a fourth embodiment of the present invention. As illustrated in FIG. 4, for embodying the present invention, acute angle edge portions 206 may be formed on second electrodes 104. In this case, due to such a pointed protrusion structure, gas discharge for generating plasma is further facilitated.

FIG. 5 is a view illustrating a structure of a plasma discharged skin treatment device 100 according to a fifth embodiment of the present invention. Referring to FIG. 5, the plasma discharged skin treatment device 100 according to the fifth embodiment of the present invention includes a first insulator 101 forming an outer layer of the plasma discharged skin treatment device 100, a first electrode 102 formed on one surface of the first insulator 101 to serve as an electrode layer, a second insulator 103 formed on one surface of the first electrode 102 to serve as an insulation layer, a second electrode 104 formed on one surface of the second insulator 103 to serve as another electrode layer, and a third insulator 105 formed on one surface of the second electrode 104 to serve as another insulation layer, wherein a plasma is generated by a power applied between the first electrode 102 and the second electrode 104.

Meanwhile, as illustrated in FIG. 5, for embodying the present invention, a layered structure including the third insulator 105, the second electrode 104, and the second insulator 103 has a plurality of holes 303 penetrating therethrough. By forming such a plurality of holes, the second electrode 104 may serve as a porous electrode layer in which the plurality of holes 303 are formed. As described above, the plasma is discharged to an outside through the porous holes 303 provided in the second electrode 104, so as to directly act on the skin of the user.

In addition, the plurality of holes 303 provided in the second electrode 104 may further perform a function of intercepting electromagnetic waves. Further, as illustrated in FIG. 5, when embodying the present invention, in order to stably generate plasma between the first electrode 102 and the second electrode 104, it is preferable that an impedance matcher 302 and a power supply unit 301 are installed with being connected to the first electrode 102 and the second electrode 104.

As described above, according to the present invention, it is possible to enhance effects of penetrating a cosmetic product drug such as a moisturizer, a drug for skin improvement, or a drug for acne and wrinkle improvement into the skin through skin stimulation caused by visible rays, infrared rays, plasma ions, and active species generated from the plasma.

In addition, when using the plasma discharged skin treatment device of the present invention on a skin portion of a body such as a neck, waist, arm, leg and the like, it is possible to contribute to shortening the recovery time of wound healing or a surgical mark at the corresponding skin portion.

The terminology used herein is for the purpose of describing particular embodiments only and is not intended to limit the present invention thereto. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements, and/or components, but do not preclude the presence or addition of one or more other features, integers, steps, operations, elements, components, and/or groups thereof.

While the present invention has been described with reference to the preferred embodiments and modified examples, the present invention is not limited to the above-described specific embodiments and the modified examples, and it will be understood by those skilled in the related art that various modifications and variations may be made therein without departing from the scope of the present invention as defined by the appended claims, as well as these modifications and variations should not be understood separately from the technical spirit and prospect of the present invention.

### INDUSTRIAL APPLICABILITY

The present invention may be applied to cosmetic or therapeutic skin treatment equipment, such that industrial applicability thereof may be recognized in the industrial fields related to the cosmetic or therapeutic skin treatment equipment.

1. A plasma discharged skin treatment device, comprising:
a first insulator;
a first electrode provided on one surface of the first insulator;
a second insulator provided on one surface of the first electrode;
a second electrode provided on one surface of the second insulator; and
a third insulator provided on one surface of the second electrode to face a skin of a user.

2. The plasma discharged skin treatment device according to claim 1, wherein a plasma is generated by a power applied between the first electrode and the second electrode.

3. The plasma discharged skin treatment device according to claim 1, wherein the first electrode has a protrusion formed thereon.
